(19) United States
(12) Patent Application Publication
(10) Pub. No.: US 2008/0261892 A1
(43) Pub. Date: Oct. 23, 2008

(54) Acid Addition Salts of Ac-Phscn-Nh2

(76) Inventors: Robert J. Ternansky, San Diego, CA (US); Patricia L. Gladstone, San Diego, CA (US); Andrew P. Mazar, San Diego, CA (US); Amy L. Allan, Encinitas, CA (US)

Correspondence Address: JONES DAY, 222 EAST 41ST ST, NEW YORK, NY, 10017, US

(21) Appl. No.: 11/883,584

(22) PCT Filed: Feb. 1, 2006
(86) PCT No.: PCT/US06/03658

§ 371 Date: Jun. 26, 2008

Related U.S. Application Data
(60) Provisional application No. 60/649,308, filed on Feb. 1, 2005.

(51) Int. Cl. C07K 7/06; A61K 38/08; A61P 9/00; A61P 35/00

(52) U.S. Cl. 514/17; 530/330

(57) ABSTRACT

Acid addition salts of Ac—PHSCN—NH₂, methods of making acid addition salts of Ac—PHSCN—NH₂, pharmaceutical compositions of acid addition salts of Ac—PHSCN—NH₂, methods of using acid addition salts of Ac—PHSCN—NH₂ and pharmaceutical compositions thereof to treat diseases associated with angiogenesis and aberrant vascularization and methods of preventing degradation of Ac—PHSCN—NH₂ by salt formation are provided herein.

This application claims the benefit of U.S. Provisional Application No. 60/649,308, filed Feb. 1, 2005, the entirety of which is herein incorporated by reference.

### 1. FIELD OF THE INVENTION

The present invention relates generally to acid addition salts of the anti-angiogenesis peptide, Ac—PHSCN—NH₂, methods of making acid addition salts of Ac—PHSCN—NH₂, pharmaceutical compositions comprising acid addition salts of Ac—PHSCN—NH₂, methods of using acid addition salts of Ac—PHSCN—NH₂ and pharmaceutical compositions thereof to treat diseases associated with angiogenesis and aberrant vascularization and methods of preventing degradation of Ac—PHSCN—NH₂ by salt formation.

### 2. BACKGROUND OF THE INVENTION

Most forms of cancer are derived from solid tumors (Shockley et al., Ann. N. Y. Acad. Sci. 1991, 617: 367-382), which have proven resistant in the clinic to therapies such as the use of monoclonal antibodies and immunotoxins. Anti-angiogenic therapy for the treatment of cancer was developed from the recognition that solid tumors require angiogenesis (i.e., new blood vessel formation) for sustained growth (Folkman, Ann. Surg. 1972, 175: 409-416; Folkman, Mol. Med. 1995, 1(2): 120-122; Folkman, Breast Cancer Res. Treat. 1995, 36(2): 109-118; Hanahan et al., Cell 1996, 86(3): 353-364). Efficacy of anti-angiogenic therapy in animal models has been demonstrated (Millauer et al., Cancer Res. 1996, 56:1615-1620; Borgstrom et al., Prostrate 1998, 35:1-10; Benjamin et al., J. Clin. Invest. 1999, 103: 159-165; Merajver et al., Proceedings of Special AACR Conference on Angiogenesis and Cancer 1998, Abstract #B-11, January 22-24). In the absence of angiogenesis, internal cell layers of solid tumors are inadequately nourished. Further, angiogenesis (i.e., aberrant vascularization) has been implicated in numerous other diseases (e.g., ocular neovascular disease, macular degeneration, rheumatoid arthritis, etc.).

Contrastingly, normal tissue does not require angiogenesis except under specialized circumstances (e.g., wound repair, proliferation of the internal lining of the uterus during the menstrual cycle, etc.). Accordingly, a requirement for angiogenesis is a significant difference between tumor cells and normal tissue. Importantly, the dependency of tumor cells on angiogenesis, when compared to normal cells, is quantitatively greater than differences in cell replication and cell death between normal tissue and tumor tissue, which are often exploited in cancer therapy.

Angiogenesis, in tumor cells under hypoxic conditions, can be initiated by cytokines such as vascular endothelial growth factor and/or fibroblast growth factor, which bind to specific receptors on endothelial cells in the local vasculature. The activated endothelial cells secrete enzymes which remodel the associated tissue matrix and modulate expression of adhesion molecules such as integrins. Following matrix degradation, endothelial cells proliferate and migrate toward the hypoxic tumor, which results in the generation and maturation of new blood vessels.

Ac—PHSCN—NH₂ is a peptide which effectively inhibits angiogenesis (Livant, U.S. Pat. No. 6,001,965; Livant, U.S. Pat. No. 6,472,369). However, dimerization provides an inactive form which is a significant problem when the Ac—PHSCN—NH₂ is dissolved in solution or stored as a solid. Accordingly, what is needed is a method of preventing degradation of Ac—PHSCN—NH₂ under both solution phase and solid phase conditions.

### 3. SUMMARY OF THE INVENTION

The present invention satisfies these and other needs by providing acid addition salts of Ac—PHSCN—NH₂ (SEQ ID NO. 1), methods of making acid addition salts of Ac—PHSCN—NH₂, pharmaceutical compositions comprising acid addition salts of Ac—PHSCN—NH₂, methods of using acid addition salts of Ac—PHSCN—NH₂ and pharmaceutical compositions thereof to treat diseases associated with angiogenesis and aberrant vascularization and methods of preventing degradation of Ac—PHSCN—NH₂ by salt formation.

In a first aspect, acid addition salts of the anti-angiogenesis peptide Ac—PHSCN—NH₂ are provided. In some embodiments, the acid is selected from the group consisting of hydrochloric acid, methanesulfonic acid, acetic acid, glycolic acid, sulfuric acid, (+) camphorsulfonic acid, mandelic acid, salicyclic acid, succinic acid, hydrobromic acid, nitric acid and phosphoric acid. In a preferred embodiment, the acid is hydrochloric acid. In certain embodiments, the acid addition salts of Ac—PHSCN—NH₂ are purified. In other embodiments, the acid addition salts of Ac—PHSCN—NH₂ are lyophilized.

The present invention also provides acid addition salt solutions of Ac—PHSCN—NH₂ which are greater than about 85% monomer after 600 hours at 23-25° C. In one embodiment, the acid addition salt solution is pure, for example, greater than 99% pure, after more than 800 hours at 23-25° C.

In a second aspect, pharmaceutical compositions comprising acid-addition salts of the anti-angiogenesis peptide Ac—PHSCN—NH₂ are provided. The pharmaceutical compositions generally comprise an acid addition salt of Ac—PHSCN—NH₂ and a pharmaceutically acceptable vehicle, including a diluent, carrier, or excipient. The choice of diluent, carrier, and excipient will depend upon, among other factors, the desired mode of administration.

In a third aspect, the present invention provides methods for treating or preventing diseases or disorders characterized by aberrant vascularization or aberrant angiogenesis. The methods generally involve administering to a patient in need of such treatment or prevention a therapeutically effective amount of a salt of Ac—PHSCN—NH₂ which may be in a pharmaceutical composition. The methods may further comprise administering a therapeutically effective amount of an anti-angiogenic agent that is not an acid addition salt of Ac—PHSCN—NH₂. In certain embodiments, the disease or disorder to be treated is cancer, for example, breast cancer, renal cancer, brain cancer, colon cancer, prostrate cancer, chondrosarcoma or angiosarcoma. In another embodiment, the disease to be treated is Crohn's disease.

In a fourth aspect, the present invention provides kits comprising a container containing an acid addition salt of Ac—PHSCN—NH₂. In one embodiment, the acid addition salt of Ac—PHSCN—NH₂ is lyophilized. In certain embodiments, the kits further comprise a container containing a sterile aqueous solution. In other embodiments, the kits further comprise a container containing an anti-angiogenic acid that is not an acid addition salt of Ac—PHSCN—NH₂. The kits may also include a syringe and/or instructions.

### 4. BRIEF DESCRIPTION OF THE DRAWINGS

FIG. 1 is a schematic representation of the synthesis of the Ac—PHSCN—NH₂ hydrochloride salt.

FIG. 2 illustrates a solution phase comparison of the monomer versus dimer concentration as a function of time of the free base of Ac—PHSCN—NH₂ and the hydrochloride salt of Ac—PHSCN—NH₂.

FIG. 3 illustrates a solid phase comparison of the monomer versus dimer concentration as a function of time of the free base of Ac—PHSCN—NH₂ and the hydrochloride salt of Ac—PHSCN—NH₂.

FIG. 4 illustrates a solution phase comparison of the monomer versus dimer concentration as a function of time of the free base of Ac—PHSCN—NH₂, the methanesulfonic acid salt of Ac—PHSCN—NH₂, and the nitric acid salt of Ac—PHSCN—NH₂.

FIG. 5 illustrates a solid phase comparison of the monomer versus dimer concentration as a function of time of the free base of Ac—PHSCN—NH₂, the methanesulfonic acid salt of Ac—PHSCN—NH₂, and the nitric acid salt of Ac—PHSCN—NH₂.

### 5. DETAILED DESCRIPTION OF THE INVENTION

Reference will now be made in detail to embodiments of the invention. While the invention will be described in conjunction with these embodiments, it will be understood that it is not intended to limit the invention to those embodiments. To the contrary, it is intended to cover alternatives, modifications, and equivalents as may be included within the spirit and scope of the invention as defined by the appended claims.

5.1 Salts of Ac—PHSCN—NH₂

It has been discovered that formation of acid addition salts of Ac—PHSCN—NH₂ significantly protect this peptide from degradation. Accordingly, presented herein are acid addition salts of Ac—PHSCN—NH₂, methods of making acid addition salts of Ac—PHSCN—NH₂, pharmaceutical compositions comprising acid addition salts of Ac—PHSCN—NH₂, methods of using acid addition salts of Ac—PHSCN—NH₂ and pharmaceutical compositions thereof to treat diseases associated with angiogenesis and aberrant vascularization and methods of preventing degradation of Ac—PHSCN—NH₂ by salt formation.

The peptide to be formulated is preferably pure, or essentially pure and, desirably, essentially homogeneous (i.e., free from contaminating peptides or proteins, etc.). “Essentially pure” means a peptide preparation wherein at least 90% by weight is the peptide based on total weight of the preparation, preferably at least 95% by weight. An “essentially homogeneous” preparation means a peptide preparation comprising at least 99% by weight of peptide, based on total weight of the peptide in the preparation.

The acid addition salts of Ac—PHSCN—NH₂ may be formed from both organic and inorganic acids. Exemplary organic acids include generally, carboxylic acids and sulfonic acids, such as acetic acid, propionic acid, hexanoic acid, cyclopentanepropionic acid, glycolic acid, pyruvic acid, lactic acid, glycolic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, fumaric acid, oxalic acid, lactic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, t-butylacetic acid, lauryl sulfuric acid, gluconic acid, glutamic acid, hydroxynaphthoic acid, salicylic acid, stearic acid and muconic acid. Other organic acids are also known to the skilled artisan. In some embodiments, the acid addition salt of Ac—PHSCN—NH₂ is formed from methanesulfonic acid, acetic acid, glycolic acid, (+) camphorsulfonic acid, mandelic acid, salicyclic acid, succinic acid or combinations thereof.

Exemplary inorganic acids include hydrofluoric acid, perchloric acid, hydrochloric acid, hydrobromic acid, nitric acid, phosphoric acid, hydroiodic acid, chloric acid, thiocyanic acid, hypophosphorus acid, nitrous acid, cyanic acid, chromic acid, sulfurous acid, phosphorous acid or hydrazoic acid. Other inorganic acids are known to those of skill in the art. In some embodiments, the acid addition salt of Ac—PHSCN—NH₂ is formed from hydrobromic acid, nitric acid, hydrochloric acid, phosphoric acid or combinations thereof. In other embodiments, the acid addition salt of Ac—PHSCN—NH₂ is formed from hydrochloric acid.

Generally, the acid addition salts of Ac—PHSCN—NH₂ may be made by any conventional method known to those of skill in the art. These methods include saturating solutions of Ac—PHSCN—NH₂ with gaseous acids, adding solutions of acids to solutions of Ac—PHSCN—NH₂, etc. In some embodiments, an acid addition salt of Ac—PHSCN—NH₂ is made by adding slightly more than 1 equivalent (e.g., 1.05 equivalents) of the acid to a solution of Ac—PHSCN—NH₂ dissolved in distilled water. The acid addition salt is typically isolated as a solid from the aqueous mixture.

Acid addition salts of Ac—PHSCN—NH₂ can be considerably more stable than the free base in both the solid and solution phase. Without wishing to be bound by theory, the acid addition salt is believed to prevent oxidative dimerization of Ac—PHSCN—NH₂ mediated by cysteine. Acid addition salts which prevent degradation of Ac—PHSCN—NH₂ are formed from, for example, methanesulfonic acid, acetic acid, glycolic acid, sulfuric acid, (+) camphorsulfonic acid, mandelic acid, salicyclic acid, succinic acid, hydrobromic acid, nitric acid and phosphoric acid. In some embodiments, the acid addition salt is formed from hydrochloric acid.

The differences in stability between the free base of Ac—PHSCN—NH₂ and an acid addition salt of Ac—PHSCN—NH₂ (e.g., HCl) can be quite significant in both the solution phase and the solid phase. In some embodiments, the acid addition salt of Ac—PHSCN—NH₂ (e.g., HCl) is greater than about 85% monomer in solution after more than 500 hours at room temperature. In contrast, the free base of Ac—PHSCN—NH₂ has been completely converted to other products (e.g., the dimer) during the same period of time. Similarly in the solid phase, the acid addition salt of Ac—PHSCN—NH₂ (e.g., HCl) is greater than about 99% pure after more than 800 hours at room temperature, i.e., 23-25° C., while the free base is greater than about 82% pure after the same amount of time.

5.2 Assays

Those of skill in the art will appreciate that the in vitro and in vivo assays useful for measuring the anti-angiogenic activity of the salts of Ac—PHSCN—NH₂ described herein are illustrative rather than comprehensive, and modifications that can be used also will be apparent to the skilled artisan.

5.2.1 Assay for Endothelial Cell Migration

For endothelial cell (EC) migration, transwells are coated with type I collagen (50 μg/mL) by adding 200 μL of the collagen solution per transwell, then incubating overnight at 37° C. The transwells are assembled in a 24-well plate and a chemoattractant (e.g., Fibroblast Growth Factor-2 [FGF-2]) is added to the bottom chamber in a total volume of 0.8 mL media. ECs, such as human umbilical vein endothelial cells (HUVEC), which have been detached from monolayer culture using trypsin, are diluted to a final concentration of about 10⁶ cells/mL with serum-free media and 0.2 mL of this cell suspension is added to the upper chamber of each transwell. Salts of Ac—PHSCN—NH₂ may be added to both the upper and lower chambers and the migration is allowed to proceed for 5 hrs in a humidified atmosphere at 37° C. The transwells are removed from the plate stained using DIFFQUIK®, a Giemasa stain (Dade Behring, Deerfield, Ill.). Cells which do not migrate are removed from the upper chamber by scraping with a cotton swab and the membranes are detached, mounted on slides, and counted under a high-power field (400×) to determine the number of cells migrated.

5.2.2 Biological Assay of Anti-Invasive Activity

The ability of cells such as ECs or tumor cells (e.g., PC-3 human prostatic carcinoma) cells to invade through a reconstituted basement membrane (MATRIGEL™, a solubulized basement membrane preparation extracted from EHS mouse sarcoma, BD Biosciences, San Jose, Calif.) in an assay known as a MATRIGEL™ invasion assay system has been described in detail in the art (Kleinman et al., Biochemistry 1986, 25: 312-318; Parish et al., 1992, Int. J. Cancer 52:378-383). MATRIGEL™ is a reconstituted basement membrane containing type IV collagen, laminin, heparan sulfate proteoglycans such as perlecan, which bind to and localize bFGF, vitronectin as well as transforming growth factor-β (TGFβ), urokinase-type plasminogen activator (uPA), tissue plasminogen activator (tPA) and the serpin known as plasminogen activator inhibitor type 1 (PAI-1) (Chambers et al., Canc. Res. 1995, 55:1578-1585,). It is accepted in the art that results obtained in this assay for compounds which target extracellular receptors or enzymes are predictive of the efficacy of these compounds in vivo (Rabbani et al., Int. J. Cancer 1995, 63: 840-845).

Such assays employ transwell tissue culture inserts. Invasive cells are defined as cells which are able to traverse through the MATRIGEL™ and upper aspect of a polycarbonate membrane and adhere to the bottom of the membrane. Transwells (COSTAR®, Corning Life Sciences, Corning, N.Y.) containing polycarbonate membranes (8.0 μm pore size) are coated with MATRIGEL™, which has been diluted in sterile PBS to a final concentration of 75 μg/mL (60 μL of diluted MATRIGEL™ per insert), and placed in the wells of a 24-well plate. The membranes are dried overnight in a biological safety cabinet, then rehydrated by adding 100 μL of DMEM (GIBCO®, Invitrogen Corporation, Carlsbad, Calif.) containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DIFFQUIK® and the upper chamber is then scraped using a cotton swab to remove the Matrigel® and any cells which did not invade through the membrane. The membranes are detached from the transwell using an X-ACTO® blade, mounted on slides using PERMOUNT®, a toluene-based synthetic resin mounting medium (Biomeda, Foster City, Calif.), and cover-slips, then counted under a high-powered (400×) field. An average of the cells invaded is determined from 5-10 fields counted and plotted as a function of Ac—PHSCN—NH₂ salt concentration.

5.2.3 Tube-Formation Assays of Anti-Angiogenic Activity

Endothelial cells, for example, human umbilical vein endothelial cells (HUVEC) or human microvascular endothelial cells (HMVEC) which can be prepared or obtained commercially, are mixed at a concentration of 2×10⁵ cells/mL with fibrinogen (5 mg/mL in phosphate buffered saline (PBS) in a 1:1 (v/v) ratio). Thrombin is added (5 units/mL final concentration) and the mixture is immediately transferred to a 24-well plate (0.5 mL per well). The fibrin gel is allowed to form and then VEGF (vascular endothelial growth factor) and bFGF (basic fibroblast growth factor) are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch). This assay is recognized in the art to be predictive of angiogenic (or anti-angiogenic) efficacy in vivo (Min et al., Cancer Res. 1996, 56: 2428-2433,).

In an alternate assay, endothelial cell tube formation is observed when endothelial cells are cultured on MATRIGEL™ (Schnaper et al., J. Cell. Physiol. 1995, 165:107-118). Endothelial cells (1×10⁴ cells/well) are transferred onto MATRIGEL™-coated 24-well plates and tube formation is quantitated after 48 hrs. Inhibitors are tested by adding them either at the same time as the endothelial cells or at various time points thereafter. Tube formation can also be stimulated by adding (a) angiogenic growth factors such as bFGF or VEGF, (b) differentiation stimulating agents (e.g., PMA [phorbol 12-myristate 13-acetate]) or (c) a combination of these.

While not wishing to be bound by theory, this assay models angiogenesis by presenting to the endothelial cells a particular type of basement membrane, namely the layer of matrix which migrating and differentiating endothelial cells might be expected to first encounter. In addition to bound growth factors, the matrix components found in MATRIGEL™ (and in basement membranes in situ) or proteolytic products thereof may also be stimulatory for endothelial cell tube formation which makes this model complementary to the fibrin gel angiogenesis model previously described (Blood et al., Biochim. Biophys. Acta 1990, 1032:89-118; Odedrat al., Pharmac. Ther. 1991, 49:111-124,).

5.2.4 Assays for Inhibition of Proliferation

The ability of the compounds of the invention to inhibit the proliferation of EC's may be determined in a 96-well format. Type I collagen (gelatin) is used to coat the wells of the plate (0.1-1 mg/mL in PBS, 0.1 mL per well for 30 minutes at room temperature). After washing the plate (3× w/PBS), 3-6,000 cells are plated per well and allowed to attach for 4 hrs (37° C./5% CO₂) in Endothelial Growth Medium (EGM; Clonetics, Cambrex Corporation, East Rutherford, N.J.) or M199 media containing 0.1-2% FBS. The media and any unattached cells are removed at the end of 4 hrs and fresh media containing bFGF (1-10 ng/mL) or VEGF (1-10 ng/mL) is added to each well. Compounds to be tested are added last and the plate is allowed to incubate (37° C./5% CO₂) for 24-48 hrs. MTS (Promega, Madison, Wis.) is added to each well and allowed to incubate from 1-4 hrs. The absorbance at 490 nm, which is proportional to the cell number, is then measured to determine the differences in proliferation between control wells and those containing test compounds. A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Compounds to be tested are included in some of the wells. After 24 hrs, MTS is added to the plate and the assay developed and read as described above.

5.2.5 Caspase-3 Activity

The ability of the compounds of the invention to promote apoptosis of EC's may be determined by measuring activation of caspase-3. Type I collagen (gelatin) is used to coat a P100 plate and 5×10⁵ ECs are seeded in EGM containing 10% FBS. After 24 hours (at 37° C. in 5% CO₂) the medium is replaced by EGM containing 2% FBS, 10 ng/ml bFGF and the desired test compound. The cells are harvested after 6 hours, cell lysates prepared in 1% Triton and assayed using the EnzChek® Caspase-3 Assay Kit #1 (Molecular Probes, Invitrogen Corp., Carlsbad, Calif.) according to the manufacturer's instructions.

5.2.6 Corneal Angiogenesis Model

The protocol used is essentially identical to that described by Volpert et at., J. Clin. Invest. 1996, 98:671-679. Briefly, female Fischer rats (120-140 gms) are anesthetized and pellets (5 μl) comprised of Hydron pellets (pellets for insertion into the cornea are made by combining known amounts of CpG oligodeoxynucleotides, sucralfate (10 mg, Bulch Meditec, Vaerlose, Denmark), and hydron polymer in ethanol (120 mg/1 ml ethanol; Interferon Sciences, New Brunswick, N.J.), and applying the mixture to a 15×15 mm² piece of synthetic mesh (Sefar America, Inc., Kansas City, Mo.) as per Kenyon et al., Invest Opthalmol V is Sci 1996, 37:1625-1632), bFGF (150 nM), and the compounds to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

5.2.7 MATRIGEL™ Plug Assay

This assay is performed essentially as described by Passaniti et al., 1992, Lab 15 Invest. 67:519-528. Ice-cold MATRIGEL™ (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The MATRIGEL™ mixture is injected subcutaneously into 4-8 week-old athymic nude mice at sites near the abdominal midline, preferably 3 injections per mouse. The injected MATRIGEL™ forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the MATRIGEL™ plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-endothelial cell adhesion molecule or PECAM”) may also be used to confirm neovessel formation and microvessel density in the plugs.

5.2.8 Chick Chorioallantoic Membrane (CAM) Angiogenesis Assay

This assay is performed essentially as described by Nguyen et al., Microvascular Res. 1994, 47:3140, A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

5.2.9 In Vivo Assessment of Angiogenesis Inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel™ and then injected into the flank of a mouse following the protocol described in Section 5.2.7, above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenize with a tissue homogenizer.

5.2.10 Xenograft Model of Subcutaneous (s.c.) Tumor Growth

Nude mice are inoculated with MDA-MB-231 cells (human breast carcinoma) and Matrigel™ (1×10⁶ cells in 0.2 mL) s.c. in the right flank of the animals. The tumors are staged to 200 mm³ and then treatment with a test composition is initiated (100 μg/animal/day given q.d. i.p. [intraperitoneal]). Tumor volumes are obtained every other day and the animals are sacrificed after 2 weeks of treatment. The tumors are excised, weighed and paraffin embedded. Histological sections of the tumors are analyzed by H and E [Hematoxylin-and-Eosin], anti-CD31, Ki-67, TUNEL, and CD68 staining.

5.2.11 Xenograft Model of Metastasis

The compounds of the invention are also tested for inhibition of late metastasis using an experimental metastasis model (Crowley et al., Proc. Natl. Acad. Sci. USA 1993, 90:5021-5025). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This approach permits utilization of either of these markers (fluorescence detection of GFP or histochemical colorimetric detection of enzymatic activity) to follow the fate of these cells. Cells are injected, preferably i.v., and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected i.v. into the tail veins of nude (nu/nu) mice. Animals are treated with a test composition at 100 μg/animal/day given q.d. i.p. Single metastatic cells and foci are visualized and quantitated by fluorescence microscopy or light microscopic histochemistry or by grinding the tissue and quantitative calorimetric assay of the detectable label.

5.2.12 Inhibition of Spontaneous Metastasis In Vivo by PHSCN and Functional Derivatives

The rat syngeneic breast cancer system employs Mat BIII rat breast cancer cells (Xing et al., Int. J. Cancer 1996, 67:423-429). Tumor cells, for example, about 10⁶ suspended in 0.1 mL PBS, are inoculated into the mammary fat pads of female Fisher rats. At the time of inoculation, a 14-day Alza osmotic mini-pump (Mountain View, Calif.) is implanted intraperitoneally to dispense the test compound. The compound is dissolved in PBS (e.g., 200 mM stock), sterile filtered and placed in the minipump to achieve a release rate of about 4 mg/kg/day. Control animals receive vehicle (PBS) alone or a vehicle control peptide in the minipump. Animals are sacrificed at about day 14. In the rats treated with the compounds of the present invention, significant reductions in the size of the primary tumor and in the number of metastases in the spleen, lungs, liver, kidney and lymph nodes (enumerated as discrete foci) may be observed. Histological and immunohistochemical analysis reveal increased necrosis and signs of apoptosis in tumors in treated animals. Large necrotic areas are seen in tumor regions lacking neovascularization. Human or rabbit PHSCN and their derivatives to which ¹³¹I is conjugated (either 1 or 2 I atoms per molecule of peptide) are effective radiotherapeutics and are found to be at least two-fold more potent than the unconjugated polypeptides. In contrast, treatment with control peptides fails to cause a significant change in tumor size or metastasis.

5.2.13 LL Lewis Lung Carcinoma: Primary Tumor Growth

This tumor line arose spontaneously as carcinoma of the lung in a C57BL/6 mouse (Malave et al., J. Nat'l. Canc. Inst. 1979, 62:83-88). It is propagated by passage in C57BL/6 mice by subcutaneous (s.c.) inoculation and is tested in semiallogeneic C57BL/6×DBA/2 F₁ mice or in allogeneic C3H mice. Typically six animals per group for subcutaneously (s.c.) implant, or ten for intramuscular (i.m.) implant are used. Tumor may be implanted sc as a 2-4 mm fragment, or i.m. or s.c. as an inoculum of suspended cells of about 0.5-2×10⁶-cells. Treatment begins 24 hours after implant or is delayed until a tumor of specified size (usually approximately 400 mg) can be palpated. The test compound is administered i.p. daily for 11 days. Animals are followed by weighing, palpation, and measurement of tumor size. Typical tumor weight in untreated control recipients on day 12 after i.m. inoculation is 500-2500 mg. Typical median survival time is 18-28 days. A positive control compound, for example, cyclophosphamide at 20 mg/kg/injection per day on days 1-11 is used. Results computed include mean animal weight, tumor size, tumor weight, survival time. For confirmed therapeutic activity, the test composition should be tested in two multi-dose assays.

5.2.14 3LL Lewis Lung Carcinoma: Primary Growth and Metastasis Model

This assay is well known in the art (Gorelik et al., J. Nat'l. Canc. Inst. 1980, 65:1257-1264; Gorelik et al., Rec. Results Canc. Res. 1980, 75:20-28; Isakov et al., Invasion Metas. 2:12-32 (1982); Talmadge et al., J. Nat'l. Canc. Inst. 1982, 69:975-980; Hilgard et al., Br. J. Cancer 1977, 35:78-86). Test mice are male C57BL/6 mice, 2-3 months old. Following s.c, i.m., or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725).

Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 34 days after dorsal s.c. injection of 10⁶ cells. The day of tumor appearance and the diameters of established tumors are measured by caliper every two days. The treatment is given as one to five doses of peptide or derivative, per week. In another embodiment, the peptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution (Sigma-Aldrich, St. Louis, Mo.) and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd (iododeoxyuridine) into lung cells (Thakur et al., J. Lab. Clin. Med. 1977, 89:217 228). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd. One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints; or (2) mice are left intact as non-amputated tumor bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, nonparametric statistics such as the Mann-Whitney U-Test may be used for analysis. Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

5.3 Therapeutic Uses of Ac—PHSCN—NH₂ Salts

In accordance with the invention, a Ac—PHSCN—NH₂ salt and/or a pharmaceutical composition thereof is administered to a patient, preferably an animal, including, but not limited to a human, mammal, or non-human animal, such as a cow, horse, sheep, pig, fowl, cat, dog, mouse, rat, rabbit, guinea pig, etc., more preferably a mammal, and most preferably a human, suffering from a disease or disorder characterized by aberrant vascularization or aberrant angiogenesis. Aberrant vascularization or aberrant angiogenesis includes abnormal neovascularization such as the formation of new blood vessels, larger blood vessels, more branched blood vessels and any other mechanism, which is inappropriate or increases blood carrying capacity to a diseased tissue or site where the neovascularization aids or is necessary for the presence or progression of the disease or disorder. The Ac—PHSCN—NH₂ salt and/or pharmaceutical composition thereof may be used to treat aberrant vascularization or aberrant angiogenesis.

In some embodiments, diseases characterized by aberrant vascularization or aberrant angiogenesis include cancer (e.g., any vascularized tumor, preferably, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas)), arthritis, diabetes, arteriosclerosis, arteriovenous, malformations, corneal graft neovascularization, delayed wound healing, diabetic retinopathy, age related macular degeneration, granulations, burns, hemophilic joints, rheumatoid arthritis, hypertrophic scars, neovascular glaucoma, nonunion fractures, Osier Weber Syndrome, psoriasis, pyogenic, granuloma, retrolental fibroplasia, pterygium, scleroderma, trachoma, vascular adhesions, ocular neovascularization, parasitic diseases, hypertrophy following surgery, inhibition of hair growth, macular degeneration (including both wet and dry type), rheumatoid arthritis and osteoarthritis. In other embodiments, diseases characterized by aberrant vascularization and aberrant angiogenesis include cancer, macular degeneration, Crohn's disease and rheumatoid arthritis.

Further, in certain embodiments, a Ac—PHSCN—NH₂ salt and/or pharmaceutical compositions thereof are administered to a patient, preferably a human, as a preventative measure against various diseases or disorders characterized by aberrant vascularization or aberrant angiogenesis. Thus, the Ac—PHSCN—NH₂ salt and/or pharmaceutical compositions thereof may be administered as a preventative measure to a patient having a predisposition for a disease characterized by aberrant vascularization or aberrant angiogenesis. Accordingly, the Ac—PHSCN—NH₂ salt and/or pharmaceutical compositions thereof may be used for the prevention of one disease or disorder and concurrently treating another (e.g., preventing arthritis while treating cancer).

5.4 Therapeutic/Prophylactic Administration

Ac—PHSCN—NH₂ salts and/or pharmaceutical compositions thereof may be advantageously used in human or veterinary medicine. As previously described in Section 5.3 above, Ac—PHSCN—NH₂ salts and/or pharmaceutical compositions thereof are useful for the treatment or prevention of various diseases or disorders characterized by aberrant vascularization or aberrant angiogenesis.

When used to treat or prevent the above disease or disorders, Ac—PHSCN—NH₂ salts which may be in pharmaceutical compositions may be administered or applied singly, or in combination with other agents. The Ac—PHSCN—NH₂ salts which may be in a pharmaceutical composition may also be administered or applied singly, in combination with one or more other pharmaceutically active agents (e.g., other anti-cancer agents, other anti-angiogenic agents such as chelators as zinc, penicillamine, thiomolybdate etc.), including other acid-addition salts described herein.

An Ac—PHSCN—NH₂ salt optionally in a pharmaceutical composition comprising one or more Ac—PHSCN—NH₂ salts, may be administered orally. The Ac—PHSCN—NH₂ salts, which may be in pharmaceutical compositions, may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems (e.g., encapsulation in liposomes, microparticles, microcapsules, capsules, etc.) may be used to administer a compound and/or pharmaceutical composition of the invention. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds and/or pharmaceutical compositions of the invention into the bloodstream.

In specific embodiments, it may be desirable to administer one or more Ac—PHSCN—NH₂ salts and/or pharmaceutical compositions thereof locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of cancer or arthritis.

In certain embodiments, it may be desirable to introduce one or more Ac—PHSCN—NH₂ salts, optionally in a pharmaceutical composition, into the central nervous system by any suitable route, including intraventricular, intrathecal and epidural injection. Intraventricular injection may be facilitated by an intraventricular catheter, for example, attached to a reservoir, such as an Ommaya reservoir.

An Ac—PHSCN—NH₂ salt, optionally in a pharmaceutical composition, may also be administered directly to the lung by inhalation. For administration by inhalation, a salt and/or pharmaceutical composition thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoro ethane, carbon dioxide or any other suitable gas) may be used to deliver compounds of the invention directly to the lung.

Alternatively, a Dry Powder Inhaler (“DPI”) device may be used to administer an Ac—PHSCN—NH₂ salt, optionally in a pharmaceutical composition, to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura For example, capsules and cartridges of gelatin for use in an inhaler or insufflator may be formulated containing a powder mix of Ac—PHSCN—NH₂ salts and a suitable powder base such as lactose or starch for these systems.

Another type of device that may be used to deliver a Ac—PHSCN—NH₂ salt which may be in a pharmaceutical composition to the lung is a liquid spray device supplied, for example, by Aradigm Corporation (Hayward, Calif.). Liquid spray systems use extremely small nozzle holes to aerosolize liquid drug formulations that may then be directly inhaled into the lung.

In some embodiments, a nebulizer is used to deliver a compound of the invention which may be in a pharmaceutical composition to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics (Columbus, Ohio).

In other embodiments, an electrohydrodynamic (“EHD”) aerosol device is used to deliver an Ac—PHSCN—NH₂ salt which may be in a pharmaceutical composition to the lung. EHD aerosol devices use electrical energy to aerosolize liquid drug solutions or suspensions (see e.g., Noakes et al., U.S. Pat. No. 4,765,539). EHD aerosol devices may more efficiently deliver drugs to the lung than existing pulmonary delivery technologies.

In other embodiments, the Ac—PHSCN—NH₂ salts which may be in a pharmaceutical composition can be delivered in a vesicle, in particular a liposome (see Langer, 1990, Science, 249:1527-1533; Treat et al., in “Liposomes in the Therapy of Infectious Disease and Cancer,” Lopez-Berestein and Fidler (eds.), Liss, New York, pp. 353-365 (1989); see generally “Liposomes in the Therapy of Infectious Disease and Cancer,” Lopez-Berestein and Fidler (eds.), Liss, New York, pp. 353-365 (1989)).

In still other embodiments, the Ac—PHSCN—NH₂ salts which may be in pharmaceutical compositions can be delivered via sustained release systems, preferably, oral sustained release systems. In one embodiment, a pump may be used (see Langer, supra; Sefton, 1987, CRC Crit Ref Biomed Eng. 14:201; Saudek et a., 1989, N. Engl. J. Med. 321:574).

In still other embodiments, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al., 1989, J. Neurosurg. 71:105). In still other embodiments, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. &Prod Mfr., 1984, 5(3) 1-9). Factors affecting drug release are well known to the skilled artisan and have been described in the art (Bamba et al., Int. J. Pharm., 1979, 2:307).

In still other embodiments, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still other embodiments, osmotic delivery systems are used for oral sustained release administration (Verma et al., Drug Dev. Ind. Pharm., 2000, 26:695-708). In still other embodiments, OROS® (ALZA Corp., Mountain View, Calif.) osmotic devices are used for oral sustained release delivery devices (Theeuwes et al., U.S. Pat. No. 3,845,770; Theeuwes et al., U.S. Pat. No. 3,916,899).

In still other embodiments, a controlled-release system can be placed in proximity of the target of the compounds and/or pharmaceutical composition of the invention, thus requiring only a fraction of the systemic dose (see, e.g., Goodson, in “Medical Applications of Controlled Release,” supra, vol. 2, pp. 115-138 (1984)). Other controlled-release systems discussed in Langer, 1990, Science 249:1527-1533 may also be used.

5.5 Pharmaceutical Compositions

The present pharmaceutical compositions contain a therapeutically effective amount of one or more Ac—PHSCN—NH₂ salts, preferably in purified form, together with a suitable amount of a pharmaceutically acceptable vehicle, which so as to provide the form for proper administration to a patient. When administered to a patient, the Ac—PHSCN—NH₂ salts and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a Ac—PHSCN—NH₂ salt may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17th Edition, 1985).

For topical administration Ac—PHSCN—NH₂ salts may be formulated as solutions, gels, ointments, creams, suspensions, etc. as is well-known in the art. Systemic formulations include those designed for administration by injection, e.g., intradermal, subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In some embodiments, the Ac—PHSCN—NH₂ salts are formulated in accordance with routine procedures as a pharmaceutical composition adapted for intravenous administration to human beings. Typically, Ac—PHSCN—NH₂ salts for intravenous administration are solutions in sterile isotonic aqueous buffer. For injection, an Ac—PHSCN—NH₂ salt may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When the Ac—PHSCN—NH₂ salt is administered by infusion, it can be dispensed, for example, with an infusion bottle containing sterile pharmaceutical grade water or saline. When the Ac—PHSCN—NH₂ salt is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optional agents, for example, sweetening agents such as fructose, aspartame or saccharin; flavoring agents such as peppermint, oil of wintergreen, or cherry coloring agents and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkylene glycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM) etc. Additionally, flavoring agents, preservatives, coloring agents, bile salts, acylcarnitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

An Ac—PHSCN—NH₂ salt may also be formulated in rectal or vaginal pharmaceutical compositions such as suppositories or retention enemas, e.g., containing conventional suppository bases such as cocoa butter or other glycerides. In addition to the formulations described previously, a Ac—PHSCN—NH₂ salt may also be formulated as a depot preparation. Such long acting formulations may be administered by implantation (for example subcutaneously or intramuscularly) or by intramuscular injection. Thus, for example, an Ac—PHSCN—NH₂ salt may be formulated with suitable polymeric or hydrophobic materials (for example, as an emulsion in an acceptable oil) or ion exchange resins, or as sparingly soluble derivatives, for example, as a sparingly soluble salt.

Finally an Ac—PHSCN—NH₂ salt may be formulated as described in co-pending applications by Mazar et al., both entitled “Improved Formulations of Anti-Angiogenic Peptides”, U.S. Provisional Application Ser. No. 60/648,391, filed Feb. 1, 2005, and International Application No. to be assigned, filed Feb. 1, 2006 (Attorney Docket No. 28932.0013), each of which is incorporated by reference herein in its entirety. The formulations that can be used comprise an additional compound that stabilizes Ac—PHSCN—NH₂ salt against spontaneous tandem dimerization or higher oligomerization. Thus, any of the pharmaceutical compositions described in this application may further comprise one or more of such additional compounds. Preferably, the additional compound is a biocompatible acid buffer with a pK of about 5, for example, citrate, acetate or 2-(N-morpho-lino)ethanesulfonic acid (MES). The acid buffer is preferably citrate at a concentration of about 25 mM or 50 mM. The buffer may be supplemented with glycine as an excipient and bulking agent, preferably at a concentration of about 50 mg/ml. The formulation may further comprise or more reducing agents such as dithiothreitol β-mercaptoethanol, or glutathione. The formulation may also comprise a lyoprotectant present in an lyoprotecting amount, for example, about 50-600 mole lyoprotectant: 1 mole peptide. The lyoprotectant is one or more sugars (such as sucrose or trehalose), one or more amino acids (such as monosodium glutamate or histidine), one or more methylamine (such as betaine), one or more lyotropic salts (such as magnesium sulfate), and/or one or more polyols (such as glycerin, erythritol, glycerol, arabitol, xylitol, sorbitol, mannitol, and propylene glycol).

5.6 Doses

An Ac—PHSCN—NH₂ salt, or pharmaceutical compositions thereof, will generally be used in an amount effective to achieve the intended purpose. For use to treat or prevent diseases or disorders characterized by aberrant vascularization or aberrant angiogenesis, the Ac—PHSCN—NH₂ salts which may be in pharmaceutical compositions, are administered or applied in a therapeutically effective amount.

The amount of a Ac—PHSCN—NH₂ salt that will be effective in the treatment of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of an Ac—PHSCN—NH₂ salt administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. Dosing may be repeated intermittently, may be provided alone or in combination with other drugs and may continue as long as required for effective treatment of the disease state or disorder.

Suitable dosage ranges for oral administration are dependent on the potency of the drug, but are generally 0.001 mg to 200 mg, preferably 0.01 mg to 50 mg, more preferably, 0.1 to 50 mg, of a compound of the invention per kilogram body weight. Dosage ranges may be readily determined by methods known to the artisan of ordinary skill.

Suitable dosage ranges for intravenous (i.v.) administration are about 0.01 mg to about 100 mg per kilogram body weight. Suitable dosage ranges for intranasal administration are generally 0.01 mg/kg body weight to 50 mg/kg body weight or 0.10 mg/kg body weight to 10 mg/kg body weight. Suppositories generally contain about 0.01 milligram to about 50 milligrams of a compound of the invention per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kilogram of body weight. Effective doses may be extrapolated from dose-response curves derived from in vitro or animal model test systems. Such animal models and systems are well-known in the art.

In a specific embodiment, the dosage administered is not based on body weight, but is an absolute amount, for example, in the range of 1 to 1000 mg per dose. In a specific embodiment, the dosage is 10 to 750 mg per dose, e.g., 20 mg, 100 mg, or 600 mg per dose. In a specific embodiment, the dose is administered from one to several (e.g., 2, 3, 4, or 7) times a week.

The Ac—PHSCN—NH₂ salts are preferably assayed in vitro and in vivo, as described above for the desired therapeutic or prophylactic activity, prior to use in humans. For example, in vitro assays can be used to determine whether administration of an Ac—PHSCN—NH₂ salt or a combination of Ac—PHSCN—NH₂ salts is preferred for treating diseases characterized by aberrant vascularization. The Ac—PHSCN—NH₂ salts may also be demonstrated to be effective and safe using animal model systems.

Preferably, a therapeutically effective dose of a Ac—PHSCN—NH₂ salt described herein will provide therapeutic benefit without causing substantial toxicity.

Toxicity of Ac—PHSCN—NH₂ salts may be determined using standard pharmaceutical procedures and may be readily ascertained by the skilled artisan. The dose ratio between toxic and therapeutic effect is the therapeutic index. An Ac—PHSCN—NH₂ salt will preferably exhibit particularly high therapeutic indices in treating disease and disorders. The dosage of an Ac—PHSCN—NH₂ salt described herein will preferably be within a range of circulating concentrations that include an effective dose with little or no toxicity.

5.7 Combination Therapy

In certain embodiments, the Ac—PHSCN—NH₂ salts which may be in a pharmaceutical compositions can be used in combination therapy with at least one other therapeutic agent that is not an acid-addition salt of Ac—PHSCN—NH₂, e.g., is not the acid-addition salt used in combination. The Ac—PHSCN—NH₂ salt, which may be in a pharmaceutical composition, and the therapeutic agent can act additively or, more preferably, synergistically. In some embodiments, an Ac—PHSCN—NH₂ salt, optionally in a pharmaceutical composition, is administered concurrently with the administration of another therapeutic agent. In other embodiments, an Ac—PHSCN—NH₂ salt or a pharmaceutical composition thereof is administered prior or subsequent to administration of another therapeutic agent. The administration of the two agents can be separate, or together in the same composition. In a specific embodiment, the other therapeutic agent is an anti-angiogenic agent or a chemotherapeutic agent.

In specific embodiments, the Ac—PHSCN—NH₂ salts, which may be in pharmaceutical compositions can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines) antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxorubicin, bleomycin, mithrmycin, mitomycin C, L-asparaginase, interferon alpha), platinum coordination complexes (e.g., cis-platinum, carboplatin, etc.), mitoxantrone, hydroxyurea, procarbazine, hormones and antagonists (e.g., prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, etc.), anti-angiogenesis agents or inhibitors (e.g., angiostatin, retinoic acids and paclitaxel, estradiol derivatives, thiazolopyrimidine derivatives, etc.), apoptosis-inducing agents (e.g., antisense oligonucleotides that block oncogenes which inhibit apoptosis, tumor suppressors, TRAIL, TRAIL polypeptide, Fas-associated factor 1, interleukin-1β-converting enzyme, phosphotyrosine inhibitors, RXR retinoid receptor agonists, carbostyril derivatives, etc.) and chelators (penicillamine, zinc, trientine, etc.)).

5.8 Therapeutic Kits

The current invention provides therapeutic kits comprising in a container the Ac—PHSCN—NH₂ salt or pharmaceutical composition thereof. The therapeutic kits may also contain one or more other compounds (e.g., chemotherapeutic agents, natural products, hormones or antagonists, anti-angiogenesis agents or inhibitors, apoptosis-inducing agents or chelators) or pharmaceutical compositions of these other compounds, in the same or separate containers.

Therapeutic kits may have a single container which contains the Ac—PHSCN—NH₂ salt or pharmaceutical compositions thereof with or without other components (e.g., other compounds or pharmaceutical compositions of these other compounds) or may have distinct containers for each component. Preferably, therapeutic kits of the invention include an Ac—PHSCN—NH₂ salt or a pharmaceutical composition thereof packaged for use in combination with the co-administration of a second compound (preferably, a chemotherapeutic agent, a natural product, a hormone or antagonist, a anti-angiogenesis agent or inhibitor, an apoptosis-inducing agent or a chelator) or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, etc.), which enables administration of other components of the kit.

### 6. EXAMPLES

The invention is further defined by reference to the following examples, which describe in detail, preparation of Ac—PHSCN—NH₂ salts and methods for measuring stability. It will be apparent to those skilled in the art that many modifications, both to materials and methods, may be practiced without departing from the scope of the invention.

### 6.1 Example 1

### Preparation of Ac—PHSCN—NH₂ Hydrochloride Salt

### Preparation of Ac-Pro-His-Ser-Cys-Asn-NH₂, TFA salt

Rink Amide AM resin (Novabiochem, EMD Biosciences, Inc., San Diego, Calif.) was treated with 20% piperidine in DMF (dimethylformamide; 1 mL per 100 mg of resin) for three minutes with nitrogen agitation and the reaction mixture was filtered and washed once with DMF. This step was repeated an additional two times. The resin was washed three times with DMF and three times with dichloromethane. Fmoc-Asn(trt)-OH (3 eq), HBTU (3 eq), and HOBt (3 eq) were dissolved in DMF (1 mL per 100 mg of resin) and added to the above resin, followed by the addition of N-methylmorpholine (NMM) (6 eq) and the mixture was agitated for 1 hour. The reaction mixture was filtered and the resin was washed three times with DMF and three times with dichloromethane. This coupling step was repeated. The Fmoc deprotection and the coupling steps described above were sequentially used with Fmoc-Cys(trt)-OH, Fmoc-Ser(trt)-OH and Fmoc-His(trt)-OH to afford Fmoc-His(trt)-Ser(trt)-Cys(trt)-Asn(trt) bound to the resin. This resin was treated with 20% piperidine in DMF (1 mL per 100 mg of resin) for three minutes with nitrogen agitation and the reaction mixture was filtered and washed once with DMF. This step was repeated an additional two times. The resin was washed three times with DMF and three times with dichloromethane. Ac-Pro-OH (3 eq), HBTU (3 eq), and HOBt (3 eq) were dissolved in DMF (1 mL per 100 mg of resin) and added to the above resin, followed by the addition of N-methylmorpholine (NMM) (6 eq) and the mixture was agitated for 1 hour. The reaction mixture was filtered and the resin was washed three times with DMF and three times with dichloromethane to afford Rink Amide AM resin bound Ac-Pro-His(trt)-Ser(trt)-Cys(trt)-Asn(trt). The resin was treated with TFA/TIS/water (95:2.5:2.5, 1 mL per 100 mg of resin) and agitated with nitrogen for 2 hours. The reaction mixture was filtered, and the resin was washed once with TFA/TIS/water and three times with dichloromethane. The solvent was removed in vacuo and the resulting residue was triturated three times with ether to afford crude Ac-Pro-His-Ser-Cys-Asn-NH₂, TFA salt.

Using this general procedure, 708 mg of crude Ac—PHSCN—NH₂, TFA salt was prepared from 2 grams of Rink Amide AM resin (loading: 0.63 mmol/g).

### Purification of Ac-Pro-His-Ser-Cys-Asn-NH₂, TFA salt

The crude peptide, dissolved in a minimum amount of methanol and water, was purified by preparative reverse phase HPLC (Beckman) with a Phenomenex Synergi hydro-RP C18 column (250 mm×21.2 mm). The peptide was eluted using a gradient from 3-100% B over 30 min with a flow rate of 20 mL/min, where solvent A was water containing 0.1% trifluoroacetic acid and solvent B was acetonitrile containing 0.1% trifluoroacetic acid. Detection was at 220 nm. Fractions >95% pure by analytical HPLC analysis, Phenomenex hydro RP (250 mm×4.6 mm) using gradient 3-100% B, (Waters) were combined, concentrated to a volume of about 2-4 ml by rotary evaporation, and lyophilized. Samples were redissolved in water and transferred to a tared 2 dram vial and lyophilized a second time.

Using this method, 140 mg of pure Ac—PHSCN—NH₂, TFA salt was obtained from 338 mg of crude material: ES MS m/z (M+H)⁺ 598.2; HPLC: 99% pure.

### Ac-Pro-His-Ser-Cys-Asn-NH₂

Ac-Pro-His-Ser-Cys-Asn-NH₂, TFA salt (140 mg, 0.197 mmol) was dissolved in 2 mL of distilled water and Amberlyst A-26 (OH) resin (4.2 meq/g, 273 mg, 5.8 eq) was added. The reaction mixture was stirred at room temperature for 5 minutes. The aqueous solution was decanted, the resin was washed twice with distilled water, and the combined aqueous layers were lyophilized to afford 81 mg (69%) of Ac—PHSCN—NH₂ as a fluffy, white solid: ES MS m/z (M⁺+H)⁺ 598.2; HPLC: 94% monomer, 6% dimer.

### Ac-Pro-His-Ser-Cys-Asn-NH₂, hydrochloride salt

Ac-Pro-His-Ser-Cys-Asn-NH₂ (77 mg, 0.13 mmol) was dissolved in 3 mL of distilled water at room temperature and 1 M hydrochloric acid (0.13 mL, 0.13 mmol) was added immediately. The mixture was swirled once and then frozen and lyophilized to afford Ac—PHSCN—NH₂, hydrochloride salt as a fluffy white solid: ¹H NMR (300 MHz, DMSO-d6) δ 9.00 (s, 1H), 8.57-8.26 (m, 2H), 8.21-8.03 (m, 2H), 7.45-7.36 (m, 2H), 7.13 (s, 1H), 7.09 (s, 1H), 6.94 (s, 1H), 4.79-4.59 (m, 1H), 4.50-4.25 (m, 4H), 3.74-3.56 (m, 3H, overlapping with water peak), 3.25-3.15 (m, 2H, overlapping with water peak), 3.11-2.98 (m, 1H), 2.88-2.72 (m, 2H), 2.57-2.38 (m, 2H, overlapping with DMSO peak), 2.02 (s, 3H), 1.92-1.65 (m, 4H); HPLC: 93% monomer, 7% dimer.

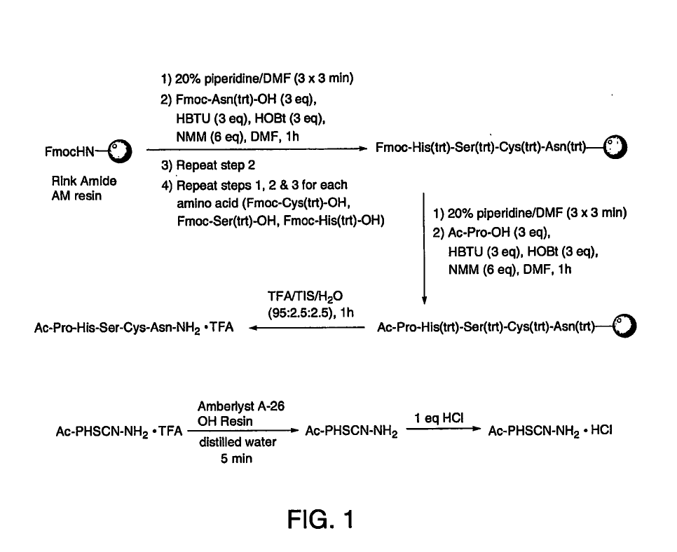

The procedure for synthesis of Ac—PHSCN—NH₂ is shown in FIG. 1.

### 6.2 Example 2

### Preparation of Ac—PHSCN—NH₂ Free Base

Ac-Pro-His-Ser-Cys-Asn-NH₂ hydrochloride salt (301 mg, 0.475 mmol) was dissolved in 4.7 mL of distilled water and Amberlyst A-26 (OH) resin (4.2 meq/g, 623 mg, 5.5 eq) was added. The reaction mixture was stirred at room temperature for 5 minutes. The aqueous solution was decanted, the resin was washed twice with distilled water, and the combined aqueous layers were lyophilized to afford 139 mg (49%) of the title compound as a fluffy, white solid: ¹H NMR (300 MHz, DMSO d6) δ 8.56 (br s, 1H), 8.23-7.84 (m, 3H), 7.59-7.52 (m, 1H), 7.33 (s, 1H), 7.12 (s, 1H), 7.07-7.00 (m, 1H), 6.97-6.77 (m, 2H), 5.19-4.98 (m, 1H), 4.62-4.16 (m, 5H), 3.70-3.51 (m, 3H), 3.03-2.72 (m, 4H), 2.53-2.36 (m, 2H, overlapping with DMSO peak), 2.00 (s, 3H), 1.90-1.75 (m, 2H), 1.74-1.63 (m, 2H); ES MS m/z (M+H)⁺ 598.3; Anal. calcd for C₂₃H₃₅N₉O₈S: N, 21.09. Found: 19.66 (peptide content, 93%). Chlorine content (IC): 0.01%.

### 6.3 Example 3

### Preparation of Various Salt Forms of Ac—PHSCN—NH₂ Free Base

Ac-Pro-His-Ser-Cys-Asn-NH₂ was dissolved in distilled water (1 mL per 20 mg of peptide) at room temperature and the appropriate acid (1.05 eq) was added immediately. The mixture was swirled once and then frozen and lyophilized to afford a fluffy, white solid.

### 6.4 Example 4

### Ac-Pro-His-Ser-Cys-Asn-NH₂ Methanesulfonic Acid Salt

This compound was prepared from the free base (51 mg, 0.073 mmol) and methanesulfonic acid (5 μL, 0.077 mmol) according to the procedure of Example 3 as a fluffy, white solid (49 mg, 98%): ¹H NMR (300 MHz, DMSO-d6) δ 8.98 (s, 1H), 8.57-8.22 (m, 2H), 8.18-7.91 (m, 2H), 7.42-7.34 (m, 2H), 7.18-7.06 (m, 2H), 6.95 (s, 1H), 4.79-4.58 (m, 1H), 4.57-4.22 (m, 4H), 3.23-3.12 (m, 3H), 3.06-2.71 (m, 4H), 2.34 (s, 3H), 2.00 (s, 3H), 1.90-1.64 (m, 4H); ES MS m/z (M+H)⁺ 598.3; Anal. calcd for C₂₄H₃₉N₉0₁₁S₂: N, 21.09; S, 9.24. Found: N, 15.88 (peptide content, 87%); S, 8.40. Chlorine content (IC): 0.0002%.

### 6.5 Example 5

### Ac-Pro-His-Ser-Cys-Asn-NH₂ Acetic Acid Salt

This compound was prepared from the free base (22 mg, 0.034 mmol) and acetic acid (2.1 μL, 0.036 mmol) according to the procedure of Example 3 as a fluffy, white solid (20 mg, 87%): ¹H NMR (300 MHz, DMSO-d6) δ 8.61-8.48 (m, 1H), 8.26-7.86 (m, 3H), 7.73-7.60 (m, 1H), 7.33 (s, 1H), 7.13 (s, 1H), 7.06-6.97 (m, 1H), 6.95-6.82 (m, 2H), 4.63-4.22 (m, 5H), 3.70-3.57 (m, 4H, overlapping with water signal), 2.99-2.88 (m, 3H), 2.85-2.72 (m, 2H), 2.63-2.34 (m, 2H, overlapping with DMSO signal), 2.00 (s, 3), 1.91 (s, 3H), 1.86-1.64 (m, 4H); ES MS m/z (M+H)+598.3; 30 Chlorine content (IC): 0.09%.

### 6.6 Example 6

### Ac-Pro-His-Ser-Cys-Asn-NH₂ Glycolic Acid Salt

This compound was prepared from free base (23 mg, 0.036 mmol) and glycolic acid (2.9 mg, 0.038 mmol) according to the procedure of Example 3 as a fluffy, white solid (24 mg, 99%): ¹H NMR (300 MHz, DMSO-d6) δ 8.60-8.48 (m, 1H), 8.25-7.85 (m, 3H), 7.69-7.58 (m, 1H), 7.33 (s, 1H), 7.13 (s, 1H), 7.08-6.98 (m, 1H), 6.95-6.81 (m, 2H), 4.63-4.22 (m, 6H), 3.90 (s, 2H), 3.57-3.12 (m, 8H, overlapping with water signal), 3.04-2.87 (m, 3H), 2.84-2.72 (m, 2H), 2.65-2.37 (m, 2H, overlapping with DMSO signal), 2.00 (s, 3H), 1.89-1.64 (m, 4H); ES MS m/z (M+H)⁺ 598.3; Chlorine content (IC): 0.09%.

### 6.7 Example 7

### Ac-Pro-His-Ser-Cys-Asn-NH₂ Sulfuric Acid Salt

This compound was prepared from the free base (23 mg, 0.036 mmol) and 1.8 M sulfuric acid (21 μL, 0.038 mmol) according to the procedure of Example 3 as a fluffy, white solid (23 mg, 92%): ¹H NMR (300 MHz, DMSO-d6) δ 8.84-8.67 (m, 1H), 8.43-8.24 (m, 2H), 8.16-7.93 (m, 2H), 7.38-7.22 (m, 2H), 7.13 (s, 1H), 7.08 (s, 1H), 6.94 (s, I H), 4.77-4.57 (m, IH), 4.49-4.24 (m, 4H), 3.70-3.41 (m, 6H, overlapping with water peak), 3.18-3.06 (m, 3H, overlapping with water peak), 3.04-2.90 (m, 2H), 2.87-2.71 (m, 2H), 2.01 (s, 3H), 1.91-1.65 (m, 4H); ES MS m/z (M+H)⁺ 598.3; Anal. calcd for C₂₃H₃₇N₉O₁₂S₂: N, 18.12; S, 9.22. Found: N, 16.88 (peptide content, 93%); S, 7.76. Chlorine content (IC): 0.09%.

### 6.8 Example 8

### Ac-Pro-His-Ser-Cys-Asn-NH₂D-(+)-Camphorsulfonic Acid Salt

This compound was prepared from the free base (23 mg, 0.036 mmol) and D-(+)-camphorsulfonic acid (8.7 mg, 0.037 mmol) according to the procedure of Example 3 as a fluffy, white solid (29 mg, 100%): ¹H NMR (300 MHz, DMSO-d6) δ 8.92-8.83 (m, 1H), 8.45-8.23 (m, 2H), 8.17-7.88 (m, 2H), 7.38-7.27 (m, 2H), 7.13 (s, 1H), 7.09 (s, 1H), 6.94 (s, 1H), 5.13 (br s, 1H), 4.78-4.57 (m, 1H), 4.49-4.23 (m, 4H), 3.72-3.48 (m, 5H, overlapping with water peak), 3.20-3.09 (m, 2H, overlapping with water signal), 3.05-2.62 (m, 6H), 2.53-2.34 (m, 3H, overlapping with DMSO signal), 2.30-2.18 (m, 1H), 2.01 (s, 3H), 1.95-1.66 (m, 7H), 1.33-1.22 (m, 2H), 1.05 (s, 3H), 0.74 (s, 3H); ES MS m/z (M+H)⁺ 598.3; Chlorine content (IC): 0.09%.

### 6.9 Example 9

### Ac-Pro-His-Ser-Cys-Asn-NH₂ Mandelic Acid Salt

This compound was prepared from the free base (23 mg, 0.036 mmol) and mandelic acid (5.8 mg, 0.038 mmol) according to the procedure of Example 3 as a fluffy, white solid (30 mg, 112%): ¹H NMR (300 MHz, DMSO-d6) δ 8.61-8.49 (m, 1H), 8.25-7.86 (m, 3H), 7.68-7.57 (m, 1H), 7.44-7.25 (m, 6H), 7.13 (s, 1H), 7.07-7.00 (m, 1H), 6.96-6.81 (m, 2H), 4.99 (s, 2H), 4.63-4.20 (m, 5H), 3.54-3.10 (m, 10H, overlapping with water peak), 3.04-2.88 (m, 3H), 2.83-2.72 (m, 2H), 2.65-2.37 (m, 2H, overlapping with DMSO signal), 2.00 (s, 3H), 1.90-1.63 (m, 4H); ES MS m/z (M+H)⁺ 598.3; Chlorine content (IC): 0.01%.

### 6.10 Example 10

### Ac-Pro-His-Ser-Cys-Asn-NH₂ Salicylic Acid Salt

This compound was prepared from the free base (23 mg, 0.036 mmol) and salicylic acid (5.4 mg, 0.039 mmol) according to the procedure of Example 3 as a fluffy, white solid: ¹H NMR (300 MHz, DMSO-d6) δ 8.50-8.29 (m, 2H), 8.27-7.92 (m, 3H), 7.73 (dd, J=7.7, 1.7 Hz, 1H), 7.37-7.28 (m, 2H), 7.18-7.02 (m, 3H), 6.94 (s, 1H), 6.80-6.72 (m, 2H), 4.70-4.24 (m, 6H), 3.58-3.24 (m, 8H, overlapping with water peak), 3.12-2.89 (m, 5H), 2.84-2.71 (m, 2H), 2.66-2.37 (m, 2H, overlapping with DMSO signal), 2.00 (s, 3H), 1.92-1.63 (m, 4H); ES MS m/z (M+H)⁺ 598.3; Chlorine content (IC): 0.01%.

### 6.11 Example 11

### Ac-Pro-His-Ser-Cys-Asn-NH₂ Succinic Acid Salt

This compound was prepared from the free base (21 mg, 0.032 mmol) and succinic acid (4.1 mg, 0.035 mmol) according to the procedure of Example 3 as a fluffy, white solid (25 mg, 108%): ¹H NMR (300 MHz, DMSO-d6) δ 8.61-8.50 (m, 1H), 8.25-7.85 (m, 3H), 7.66-7.56 (m, 1H), 7.34 (s, 1H), 7.13 (s, 1H), 7.07-7.00 (m, 1H), 6.96-6.79 (m, 2H), 4.63-4.20 (m, 5H), 3.69-3.57 (m, 3H, overlapping with water peak), 3.04-2.71 (m, 5H), 2.65-2.38 (m, 2H, overlapping with DMSO signal), 2.41 (s, 4H), 2.00 (s, 3H), 1.90-1.63 (m, 4H); ES MS m/z (M+H)⁺ 598.3; Chlorine content (IC): 0.01%.

### 6.12 Example 12

### Ac-Pro-His-Ser-Cys-Asn-NH₂ Hydrobromide Salt

This compound was prepared from the free base (21 mg, 0.032 mmol) and 4.8% hydrobromic acid (38.1 μL, 0.034 mmol) according to the procedure of Example 3 as a fluffy, white solid (23 mg, 109%): ¹H NMR (300 MHz, DMSO d6) δ 9.00 (s, 1H), 8.50-8.22 (m, 2H), 8.14-7.90 (m, 2H), 7.41 (s, 1H), 7.36 (s, 1H), 7.14 (s, 1H), 7.10 (s, 1H), 6.95 (s, 1H), 4.80-4.58 (m, 1H), 4.49-4.24 (m, 4H), 3.72-3.56 (m, 3H, overlapping with water peak), 3.23-2.93 (m, 5H), 2.88-2.71 (m, 2H), 2.65-2.36 (m, 2H, overlapping with DMSO peak), 2.01 (s, 3H), 1.91-1.66 (m, 4H); ES MS m/z (M+H)⁺ 598.3; Anal. calcd for C₂₃H₃₆N₉O₈S: N, 18.58; Br, 11.78. Found: N, 16.85 (peptide content, 91%); Br, 10.80. Chlorine content (IC): 0.01%.

### 6.13 Example 13

### Ac-Pro-His-Ser-Cys-Asn-NH₂ Nitric Acid Salt

This compound was prepared from the free base (21 mg, 0.032 mmol) and 4.8% hydrobromic acid (38.1 μL, 0.034 mmol) according to the procedure of Example 3 as a fluffy, white solid (23 mg, 109%): ¹H NMR (300 MHz, DMSO-d6) δ 8.95-8.83 (m, 1H), 8.60-8.23 (m, 2H), 8.19-7.89 (m, 2H), 7.40-7.29 (m, 2H), 7.14 (s, 1H), 7.10 (s, 1H), 6.95 (s, 1H), 5.16 (br s, 1H), 4.79-4.58 (m, 1H), 4.57-4.24 (m, 4H), 3.72-3.49 (m, 5H, overlapping with water peak), 3.21-3.08 (m, 3H, overlapping with water peak), 3.05-2.88 (m, 2H), 2.86-2.71 (m, 2H), 2.64-2.37 (m, 2H, overlapping with DMSO peak), 2.01 (s, 3H), 1.93-1.57 (m, 4H); ES MS m/z (M+H)⁺ 598.3; Anal. calcd for C₂₃H₃₆N₁₀O₁₁S: C, 41.81; N, 21.20. Found: C, 40.20; N, 19.82 (peptide content, 93%). Chlorine content (IC): 0.06%.

### 6.14 Example 14

### Ac-Pro-His-Ser-Cys-Asn-NH₂ Phosphoric Acid Salt

This compound was prepared from the free base (20 mg, 0.031 mmol) and 8.5% phosphoric acid (22.1 μL, 0.032 mmol) according to the procedure of Example 3 as a fluffy, white solid (22 mg, 105%): ¹H NMR (300 MHz, DMSO-d6) δ 8.53-8.41 (m, 1H), 8.35-7.98 (m, 3H), 7.95-7.76 (m, 1H), 7.35 (s, 1H), 7.13 (s, 1H), 7.08-6.88 (m, 3H), 4.67-4.57 (m, 1H), 4.52-4.21 (m, 5H), 3.71-3.25 (m, 5H), 3.19-2.72 (m, 5H), 2.63-2.35 (m, 2H, overlapping with DMSO peak), 2.01 (s, 3H), 1.90-1.64 (m, 4H); ES MS m/z (M+H)⁺ 598.3; Anal. calcd for C₂₃H₃₆N₁₀O₁₁S: N, 18.12; P, 4.45. Found: N, 16.69 (peptide content, 92%); P, 3.94. Chlorine content (IC): 0.06%.

### 6.15 Example 15

### Comparison of the Solution Phase Stability of Ac-Pro-His-Ser-Cys-Asn-NH₂ Free Base and Hydrochloride Salt

A 1.657 mM solution of Ac—PHSCN—NH₂ in distilled water and a 1.653 mM solution of the hydrochloride salt of Ac—PHSCN—NH₂ in distilled water were placed in a water bath at 25±1° C. Aliquots (800 μL) were periodically removed and the amount of dimerization was determined by HPLC.

All HPLC spectra were taken on a Waters HPLC that included Breeze software, a Waters 2487 Dual λ Absorbance Detector, a Waters 1525 Binary HPLC Pump, and a Waters 717 plus Autosampler. The column was a Phenomenex Synergi 4μ Hydro-RP 80A, 250×4.60 mm 4μ micron. The 40 minute gradient that was used is described in the table below.

Mobile Phase A: 0.1% TFA in acetonitrile
Mobile Phase B: 0.1% TFA in water

| Time | Flow (mL/min) | % A | % B |
|---|---|---|---|
| 0 | 1.0 | 3.0 | 97.0 |
| 30.0 | 1.0 | 100 | 0 |
| 33.0 | 1.0 | 100 | 0 |
| 33.5 | 1.0 | 3.0 | 97.0 |
| 35.0 | 1.0 | 3.0 | 97.0 |

The HPLC peak for Ac-Pro-His-Ser-Cys-Asn-NH₂ monomer at approximately 9.2 minutes, and the peak for ATN-161 dimer at approximately 10.0 minutes were integrated in order to determine the percentage of these two species in each sample.

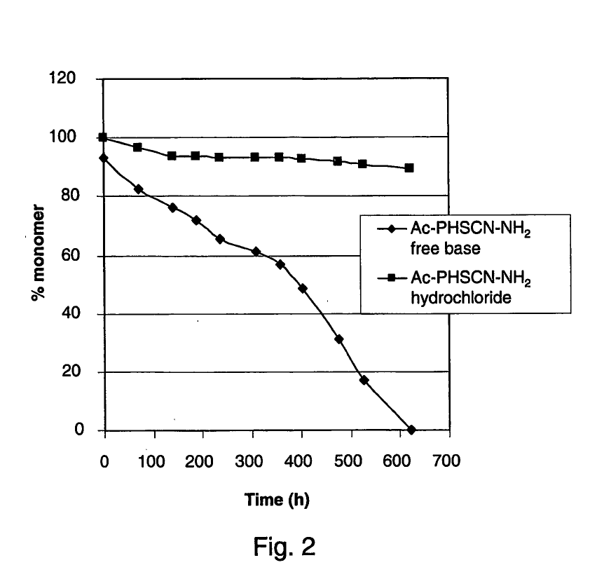

The results of the study in tabular form are as follows. FIG. 2 illustrates the results graphically.

TABLE 1
Free Base

| Time | % monomer | % dimer |
|---|---|---|
| 0 h | 93.3 | 6.7 |
| 69 h | 82.6 | 17.4 |
| 140 h | 76.5 | 23.5 |
| 188 h | 71.9 | 28.1 |
| 236 h | 65.7 | 34.3 |
| 309 h | 61.1 | 38.9 |
| 357 h | 56.7 | 43.3 |
| 404 h | 48.6 | 51.4 |
| 477 h | 31.3 | 68.7 |
| 525 h | 16.8 | 83.2 |
| 622 h | 0 | 100 |

TABLE 2
Hydrochloride salt

| Time | % monomer | % dimer |
|---|---|---|
| 0 h | 100 | 0 |
| 69 h | 96.7 | 3.3 |
| 140 h | 93.9 | 6.1 |
| 188 h | 93.6 | 6.4 |
| 236 h | 93.2 | 6.8 |
| 309 h | 93.3 | 6.7 |
| 357 h | 93.3 | 6.7 |
| 404 h | 92.7 | 7.3 |
| 477 h | 91.6 | 8.4 |
| 525 h | 91.0 | 9.0 |
| 622 h | 89.3 | 10.7 |

### 6.16 Example 16

### Comparison of the Solid Phase Stability of Ac-Pro-His Ser-Cys-Asn-NH₂ Free Base and Hydrochloride Salt

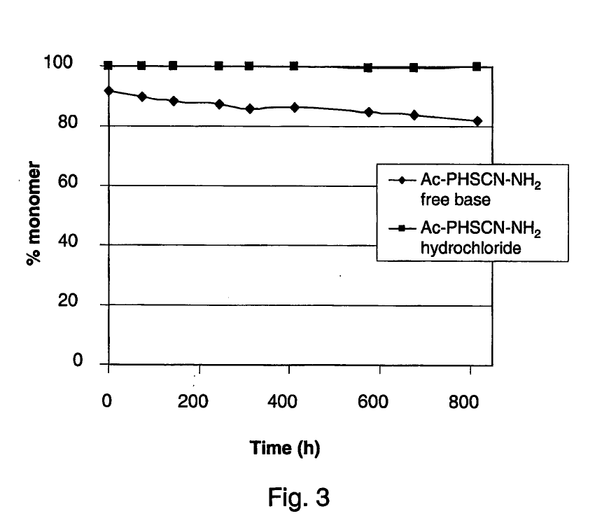

Samples of 0.8-1.2 mg of Ac—PHSCN—NH₂ and Ac—PHSCN—NH₂ hydrochloride salt were weighed into HPLC sample vials and stored at 25±1° C. A 0 h time point was taken. Samples were periodically removed and diluted with distilled water to give a 1 mg/mL solution. The amount of dimerization was determined by HPLC as described in Example 15. The results of the study in tabular form are as follows. The results are illustrated graphically in FIG. 3.

TABLE 3
Free Base

| Time | % monomer | % dimer |
|---|---|---|
| 0 h | 91.5 | 8.5 |
| 73 h | 90.0 | 10.0 |
| 143 h | 88.5 | 11.5 |
| 243 h | 87.3 | 12.7 |
| 312 h | 86.0 | 14.0 |
| 410 h | 86.2 | 13.8 |
| 576 h | 84.8 | 15.2 |
| 677 h | 83.9 | 16.1 |
| 818 h | 82.1 | 17.9 |

TABLE 4
Hydrochloride salt

| Time | % monomer | % dimer |
|---|---|---|
| 0 h | 100 | 0 |
| 73 h | 100 | 0 |
| 143 h | 100 | 0 |
| 243 h | 100 | 0 |
| 312 h | 100 | 0 |
| 410 h | 100 | 0 |
| 576 h | 99.7 | 0.3 |
| 677 h | 99.3 | 0.7 |
| 818 h | 100 | 0 |

### 6.17 Example 17

### Comparison of the Solution Phase Stability of Ac-Pro-His Ser-Cys-Asn-NH₂ Free Base, Methanesulfonic Acid Salt, and Nitric Acid Salt

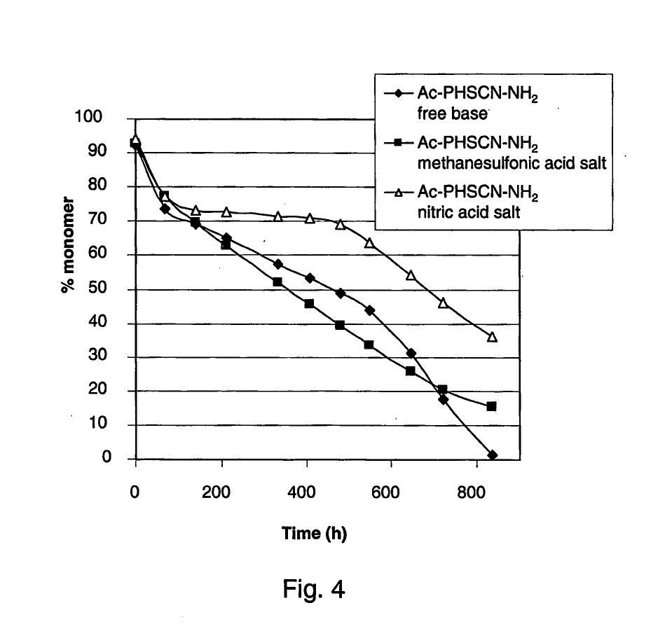

Ac—PHSCN—NH₂ methanesulfonic acid salt (31.0 mg, 93.5% monomer, 0.0418 mmol) was dissolved in distilled water in a 25 mL volumetric flask to give a 1.67 mM solution. In a second 25 mL volumetric flask, Ac—PHSCN—NH₂ nitric acid salt (29.0 mg, 93.5% monomer, 0.0410 mmol) was dissolved in distilled water to give a 1.64 mM solution, and in a third 25 mL volumetric flask, Ac—PHSCN—NH₂ free base (26.3 mg, 94.5% monomer, 0.0416 mmol) was dissolved in distilled water to give a 1.66 mM solution. A t=0 h timepoint was taken for each solution. The volumetric flasks were placed in a water bath at 25±0.5° C. and, periodically, a 0.7 mL aliquot was removed and the solution analyzed by HPLC as described in Example 15. The results of the study in tabular form are as follows. The results are illustrated graphically in FIG. 4.

TABLE 5
Free Base

| Time | % | % |
|---|---|---|
| (h) | monomer | dimer |
| 0 | 92.2 | 7.8 |
| 69 | 73.9 | 26.1 |
| 140 | 69.3 | 30.7 |
| 213 | 65.0 | 35.0 |
| 332 | 57.5 | 42.5 |
| 408 | 53.6 | 46.4 |
| 479 | 49.0 | 51.0 |
| 549 | 43.8 | 56.2 |
| 645 | 31.1 | 68.9 |
| 720 | 17.8 | 82.2 |
| 836 | 1.5 | 98.5 |

TABLE 6
methanesulfonic acid

| Time | % | % |
|---|---|---|
| (h) | monomer | dimer |
| 0 | 92.6 | 7.4 |
| 69 | 77.4 | 22.6 |
| 140 | 69.7 | 30.3 |
| 213 | 62.8 | 37.2 |
| 332 | 52.0 | 48.0 |
| 408 | 45.5 | 54.5 |
| 479 | 39.5 | 60.5 |
| 549 | 33.3 | 66.7 |
| 645 | 25.7 | 74.3 |
| 720 | 20.5 | 79.5 |
| 836 | 15.2 | 84.8 |

TABLE 7
nitric acid

| Time | % | % |
|---|---|---|
| (h) | monomer | dimer |
| 0 | 94.0 | 6.0 |
| 69 | 77.3 | 22.7 |
| 140 | 73.2 | 26.8 |
| 213 | 73.0 | 27.0 |
| 332 | 71.4 | 28.6 |
| 408 | 71.1 | 28.9 |
| 479 | 69.2 | 30.8 |
| 549 | 63.6 | 36.4 |
| 645 | 54.1 | 45.9 |
| 720 | 46.0 | 54.0 |
| 836 | 36.0 | 64.0 |

### 6.18 Example 18

### Comparison of the Solid Phase Stability of Ac-Pro-His Ser-Cys-Asn-NH₂ Free Base, Methanesulfonic Acid Salt, and Nitric Acid Salt

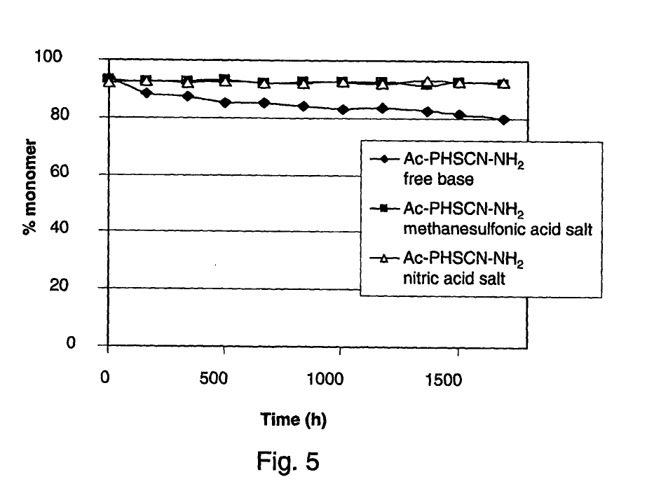

Eleven samples of Ac—PHSCN—NH₂ methanesulfonic acid salt (0.7-1.0 mg per sample), twelve samples of Ac—PHSCN—NH₂ nitric acid salt (0.7-1.0 mg per sample), and twelve samples of Ac—PHSCN—NH₂ free base (0.7-1.0 mg per sample) were weighed into HPLC sample vials, and the vials were stored in an incubator at 25±0.5° C. Periodically a sample vial of each peptide was removed and the material was dissolved in enough distilled water to give a 1 mg/ML solution, which was analyzed by HPLC as described in Example 15. The results of the study in tabular form are as follows. The results are illustrated graphically in FIG. 5.

TABLE 8
Free Base

| Time | % | % |
|---|---|---|
| (h) | monomer | dimer |
| 0 | 93.9 | 6.1 |
| 161 | 88.4 | 11.6 |
| 335 | 87.5 | 12.5 |
| 498 | 85.4 | 14.6 |
| 670 | 85.2 | 14.8 |
| 832 | 84.3 | 15.7 |
| 1003 | 83.0 | 17.0 |
| 1170 | 83.5 | 16.5 |
| 1363 | 82.4 | 17.6 |
| 1506 | 81.4 | 18.6 |
| 1698 | 80.8 | 19.2 |

TABLE 9
methanesulfonic acid

| Time | % | % |
|---|---|---|
| (h) | monomer | dimer |
| 0 | 93.2 | 6.8 |
| 161 | 92.8 | 7.2 |
| 335 | 92.5 | 7.5 |
| 498 | 93.0 | 7.0 |
| 670 | 92.3 | 7.7 |
| 832 | 92.8 | 7.2 |
| 1003 | 92.8 | 7.2 |
| 1170 | 92.7 | 7.3 |
| 1363 | 91.4 | 8.6 |
| 1506 | 92.4 | 7.6 |
| 1698 | 91.9 | 8.1 |

TABLE 10
nitric acid

| Time | % | % |
|---|---|---|
| (h) | monomer | dimer |
| 0 | 92.0 | 8.0 |
| 161 | 92.6 | 7.4 |
| 335 | 92.2 | 7.8 |
| 498 | 92.8 | 7.2 |
| 670 | 92.3 | 7.7 |
| 832 | 92.3 | 7.7 |
| 1003 | 92.6 | 7.4 |
| 1170 | 92.3 | 7.7 |
| 1363 | 93.3 | 6.7 |
| 1506 | 92.6 | 7.4 |
| 1698 | 92.4 | 7.6 |

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference.

What is claimed is:

1. An acid addition salt of Ac—PHSCN—NH₂ (SEQ ID NO. 1).

2. The acid addition salt of claim 1 in which the acid is selected from the group consisting of methanesulfonic acid, acetic acid, glycolic acid, sulfuric acid, (+) camphorsulfonic acid, mandelic acid, salicyclic acid, succinic acid, hydrobromic acid, nitric acid and phosphoric acid.

3. The acid addition salt of claim 1 in which the acid is hydrochloric acid.

4. A pharmaceutical composition comprising the acid addition salt of claim 1 and a pharmaceutically acceptable vehicle.

5. A method for treating or preventing a disease or disorder characterized by aberrant vascularization or aberrant angiogenesis in a patient comprising administering to the patient in need of such treating or preventing a therapeutically effective amount of the acid addition salt of claim 1.

6. The method of claim 5, wherein the acid addition salt is purified.

7. A method for treating or preventing a disease or disorder characterized by aberrant vascularization or aberrant angiogenesis in a patient comprising administering to the patient in need of such treating or preventing a therapeutically effective amount of the pharmaceutical composition of claim 4.

8. The method of claim 5 which is a method for treating and which further comprises administering to the patient in need of such treating a therapeutically effective amount of an anti-angiogenic agent that is not said acid addition salt.

9. The method of claim 7 which is a method for treating and which further comprises administering to the patient in need of such treating a therapeutically effective amount of an anti-angiogenic agent that is not said acid addition salt.

10. The method of claim 5, wherein the angiogenic disease is cancer.

11. The method of claim 10, wherein the cancer is breast cancer, renal cancer, brain cancer, colon cancer, prostrate cancer, chondrosarcoma or angiosarcoma.

12. The method of claim 7, wherein the angiogenic disease is cancer.

13. The method of claim 12, wherein the cancer is breast cancer, renal cancer, brain cancer, colon cancer, prostrate cancer, chondrosarcoma or angiosarcoma.

14. The method of claim 7, wherein the acid is hydrochloric acid.

15. A method of stabilizing Ac—PHSCN—NH₂ against degradation, said method comprising adding an acid to Ac—PHSCN—NH₂.

16. The method of claim 15 in which the degradation is due to dimerization

17. The method of claim 15 in which the acid is selected from the group consisting of methanesulfonic acid, acetic acid, glycolic acid, sulfuric acid, (+) camphorsulfonic acid, mandelic acid, salicyclic acid, succinic acid, hydrobromic acid, nitric acid and phosphoric acid.

18. The method of claim 15 in which the acid is hydrochloric acid.

19. A solution comprising the acid addition salt of claim 1, which is greater than 85% monomer of said salt after about 600 hours at 23-25° C.

20. The solution of claim 19 in which the acid is hydrochloric acid.

21. An acid addition salt of Ac—PHSCN—NH₂ which is essentially pure after more than 800 hours at 23-25° C.

22. The acid addition salt of claim 21 which is greater than 99% pure.

23. The acid addition salt of claim 21 in which the acid is hydrochloric acid.

24. The acid addition salt of claim 1 which is lyophilized.

25. The acid addition salt of claim 24 in which the acid is hydrochloric acid.

26. A kit comprising a first container containing an acid addition salt of Ac—PHSCN—NH₂.

27. The kit of claim 26, wherein the acid addition salt of Ac—PHSCN—NH₂ is lyophilized.

28. The kit of claim 27, further comprising a second container containing a sterile aqueous solution.

29. The kit of claim 26, further comprising a syringe.

30. The kit of claim 26, further comprising a second container containing an anti-angiogenic agent that is not the acid addition salt of Ac—PHSCN—NH₂.

31. An acid addition salt of Ac—PHSCN—NH₂ for use as a medicament.


SEQUENCE LISTING (1 sequence)

SEQ ID NO: 1 - protein, 5 aa, Artificial Sequence
  Fibronectin derivative (Anti-angiogenesis peptide)
PHSCN